(19) United States
(12) Patent Application Publication
(10) Pub. No.: US 2008/0241933 A1
(43) Pub. Date: Oct. 2, 2008

(54) Biological Saw Sensor

(75) Inventors: Jarrod Barker, Victoria (AU); Shaun Holthouse, Victoria (AU); Mathew Solomon, Victoria (AU)

Correspondence Address: CONNOLLY BOVE LODGE & HUTZ LLP, 1875 EYE STREET, N.W., SUITE 1100, WASHINGTON, DC, 20036, US

(73) Assignee: MNT Innovations Pty Ltd, Scoresby, Victoria (AU)

(21) Appl. No.: 11/572,662

(22) PCT Filed: Jul. 28, 2005
(86) PCT No.: PCT/AU05/01098

§ 371 Date: May 15, 2008

(30) Foreign Application Priority Data
Jul. 29, 2004 (AU) 2004904211

(51) Int. Cl. G01N 37/00; B01J 19/00; C12M 1/34; B01L 11/00

(52) U.S. Cl. 436/8; 422/68.1; 435/287.1; 422/101

(57) ABSTRACT

Analysing fluid samples for target biological or chemical species comprising a detector unit with a body incorporating a SAW sensor (29) for said target species, in a microfluidic channel (27) and a reader unit adapted to receive the detector unit. The reader unit includes electrical contacts with said SAW sensor and a processor to analyse the sensor signals to determine if the target is present. The detector unit includes a storage reservoir (23) for the calibrating and sample solutions arranged so that the fluid flows through the microfluidic channel over the SAW sensor to a storage reservoir. The sensor is calibrated by using a first liquid having a known quantity of a form of the target species to provide a first frequency change and then a sample solution to be measured and measuring a second frequency change and using the first and second measurements to calibrate the sensor and determine the quantity of the target in the sample solution.

This invention relates to biological sensors and in particular a sensor system that can be used to quickly and conveniently identify a variety of microorganisms or biological species.

### BACKGROUND TO THE INVENTION

The detection of pathogens or microorganism contamination as a means of diagnosis or as a means of monitoring quality of food stuffs usually involves taking a sample and conducting analysis in a laboratory. Portable analytical tools have been proposed which use microfluidic devices that can handle small volumes. Many of these use analytical processes such as PCR or bio beads. U.S. Pat. No. 6,408,878 and WO 02/081729 disclose PCR micro fluidic reactors.

USA application 2004/0115094 discloses a microfluidic analysis kit which includes a solvent and waste store on a microfluidic chip.

Various analytical tools have been proposed for identifying biological species including Matrix assisted laser desorption/ionization (MALDI) and in association with time of flight (TOF) analysis. Typical patents are U.S. Pat. Nos. 6,027,942 and 6,265,715.

WO 2004/024333 discloses a sensor comprising a set of interdigitated electrodes located in a microchannel to retain analytical biological microbeads at that location so that detection can be made by a spectroscopic method.

The use of surface acoustic wave (SAW) sensors in a microfluidic device is suggested in U.S. Pat. No. 6,553,319. This patent discloses an unattended self calibrating liquid analyzer that uses a an evacuated housing to produce regular fluid flow. WO 02/95940 discloses a saw sensor having a zinc oxide layer on a quartz substrate that generates love mode waves and provides a sensitive biological sensor. This system and most prior art systems have the problem that quantitative measurements are compromised by age of the test surface and variations in the SAW device and the bio receptor layers.

In any portable micro analytic system attention needs to be paid to the power requirements and ancillary devices needed such as pumps waste storage. USA 2003/0132112 discloses a method of pumping fluid through a micro fluidic channel by creating a pressure gradient across the channel by depositing a reservoir drop across the out put port and depositing pumping drops at the inlet port. USA 2003/0096405 by discloses a gravity driven pump for a microfluidic system.

It is an object of this invention to provide a system for detecting biological species that is portable and relatively quick to provide results as well as being relatively inexpensive to operate.

It is also an object of this invention to provide a calibration technique for devices for detecting biological species.

### BRIEF DESCRIPTION OF THE INVENTION

To this end the present invention provides a sensor system for analyzing fluid samples for the presence of a target biological or chemical species comprising a
a) a detector unit consisting of a body incorporating a surface acoustic wave (SAW) sensor for said target species, disposed in a microfluidic channel and a storage receptacle for processed fluid
b) a reader unit adapted to receive the detector unit said reader unit including means to establish electrical contact with said surface acoustic wave sensor and a processor to analyse the sensor signals to determine if the target species is in said sample.

When the SAW device interacts with a target analytes the operating frequency changes. The change of operating frequency is proportional to the magnitude of the target analyte in the environment. The combination provides for relatively inexpensive one use detector unit and a portable reader that can be used with a variety of detector units for differing target species. In a preferred aspect the reader is a small battery powered unit housing a recess for the detector unit and the electronics for the processor and a cradle for a personal digital assistant (PDA) which is used to store and display the signals received from the SAW sensor and processed by the processor. This enables the sensor system to be used in remote non laboratory locations and enables immediate on site analysis of fluid samples as well as enabling the results to be stored for more extensive analysis on a computer.
1. The SAW sensor is preferably of the layered type where a biological sensitive layer is located on the surface of the SAW device. A gold film may be deposited on the surface. Gold interacts with high affinity to proteins. It can be used with specific antibodies for antigen detection. This deposit can be made on a porous surface as well as a smooth surface. It is within the scope of this invention to have the saw electronics in the reader for inducing an acoustic wave in the surface supporting the analyte sensitive surface arranged in the detector unit. In this arrangement the surface acoustic wave sensor is incorporated in the reader unit and the detector unit incorporates a biologically sensitive surface that seats on the surface acoustic wave sensor when the detector unit is docked in the reader unit. Such an arrangement is disclosed in U.S. Pat. No. 6,626,026.

A preferred SAW sensor is disclosed in WO 02/95940 and Australian application PCT/AU2005/000244 which disclose a surface acoustic wave sensor which incorporates
a) a first layered SAW device consisting of a piezoelectric crystal with interdigital electrodes on its surface, and second piezoelectric layer over said interdigital electrodes
b) a second layered SAW device consisting of a piezoelectric crystal with interdigital electrodes on its surface, a second piezoelectric layer over said interdigital electrodes and an analyte sensitive surface on said second piezoelectric layer
c) both SAW devices are fabricated on the same substrate.

This provides a sensitive detection system for target analytes that can be made sufficiently small that it can be used in a microfluidic channel device. The SAW sensor may be treated to detect any biological target. For quality control in food production the SAW device can be treated to detect quantitatively the presence of Salmonella, E Coli, or other enteric pathogens. For environmental monitoring pathogens such as legionella can be detected. An array of sensor surfaces each prepared to detect a specific target analyte may be included in the one detector unit so that more than one pathogen may be detected from any one sample.

The detector unit is preferably arranged so that no pumping or valves are needed to move the sample through the microfluidic channel and over the surface of the SAW sensor. By arranging the reservoir of the sample above the waste reservoir gravity flow can be achieved from the sample reservoir over the SAW sensor and then to the waste reservoir.

In a further aspect the present invention provides a method of calibrating a biological sensor of the type in which a sample fluid containing a target species is brought into contact with a receptor surface containing a reagent that binds to the target species the calibration technique including the steps of contacting the receptor surface with first liquid having a known quantity of a form of the target species to provide a first measurement and then contacting the receptor with the sample solution of to be measured and taking a second measurement and using the first measurement to calibrate the sensor quantitatively to determine the quantity of the target in the sample solution from the second measurement. This technique is particularly useful in the SAW sensor disclosed in copending application PCT/AU2005/000244 the contents of which are incorporated herein by reference.

When the SAW device interacts with a target analytes the operating frequency changes. The change of operating frequency is proportional to the magnitude of the target analyte in the environment. Preferably the calibrating solution contains a known amount of an inactive form of the target species. The first frequency change generated by the calibrating solution is used to calibrate the sensor and the second frequency change is a measure of the amount of the target species in the sample. Where the sensor response is non linear a second calibration solution could be contacted with the receptor surface after the sample solution to obtain a third frequency change to obtain a further value for use in calibration.

Where a disposable sensor unit is arranged in a microfluidic surface the known quantity of the non viable form of the target species may be placed in the microfluidic channel upstream of the SAW sensor. The device may include a reservoir for distilled water which is used to flush the non viable material and flow it across the sensor to obtain the first calibrating measurement. Only the quantity of the non viable material need be known not the volume of the distilled water. This arrangement provides a longer life for the one use sensor portion as the nonviable material such as freeze dried legionella has a similar shelf life to the legionella antibodies fixed to the receptor surface. This would be superior to a prepared solution of the legionella cells which may decompose more quickly than the freeze dried specimen.

To improve the sensitivity of the method it is preferred to preconcentrate the sample by collecting the biological target species on a filter, rinsing them into concentrated sample in the detector unit. This invention provides a filter which may be connected to a pressurized source of the biological fluid to be analysed so that the biological species content of the fluid may be collected on the filter and rinsed off with a small volume of wate tp provides concentrated sample for the detector unit. Water from airconditioning cooling towers and process water from food processing plants may be sampled in this way

### DETAILED DESCRIPTION OF THE INVENTION

A preferred embodiment of the invention is described with reference to the drawings in which:

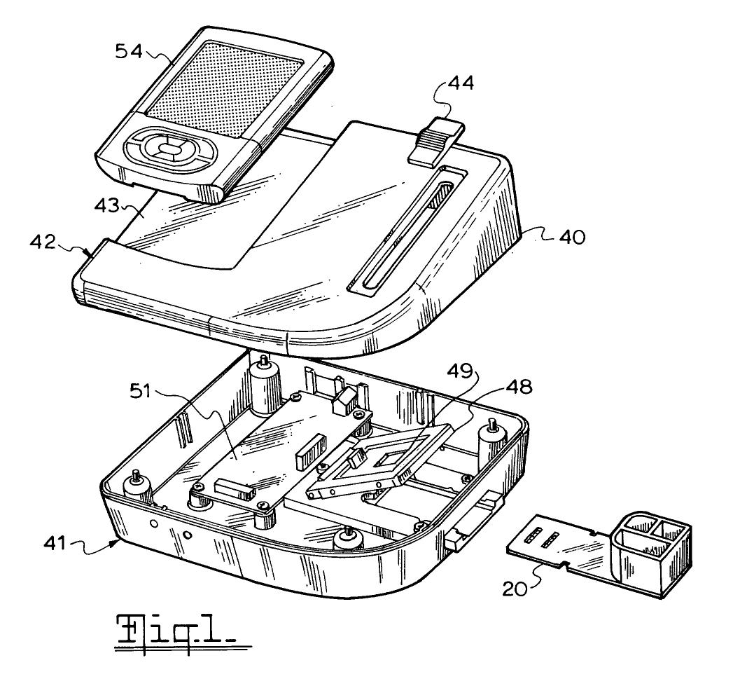

FIG. 1 illustrates an exploded diagram of the detector unit and the associated reader;

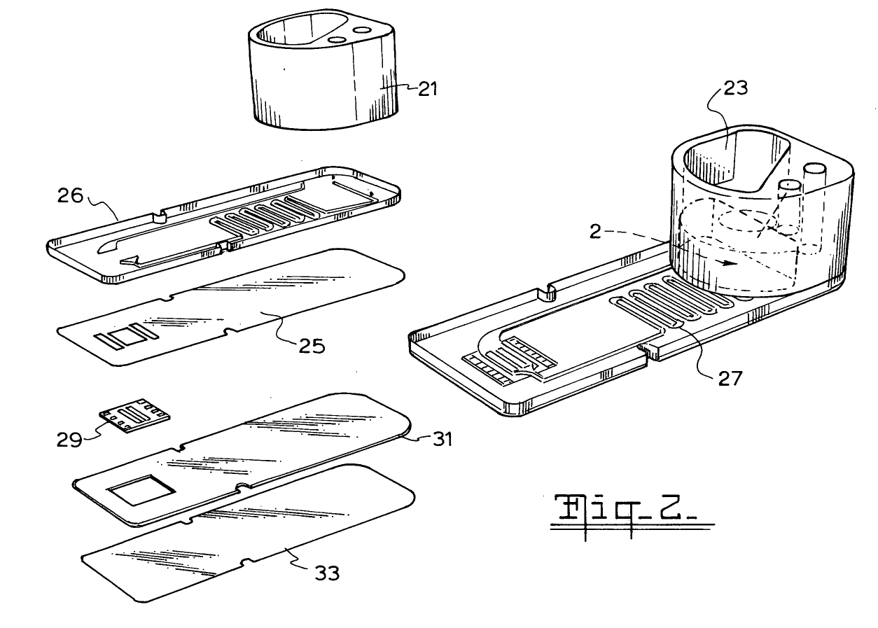

FIG. 2 illustrates an exploded view of the detector unit;

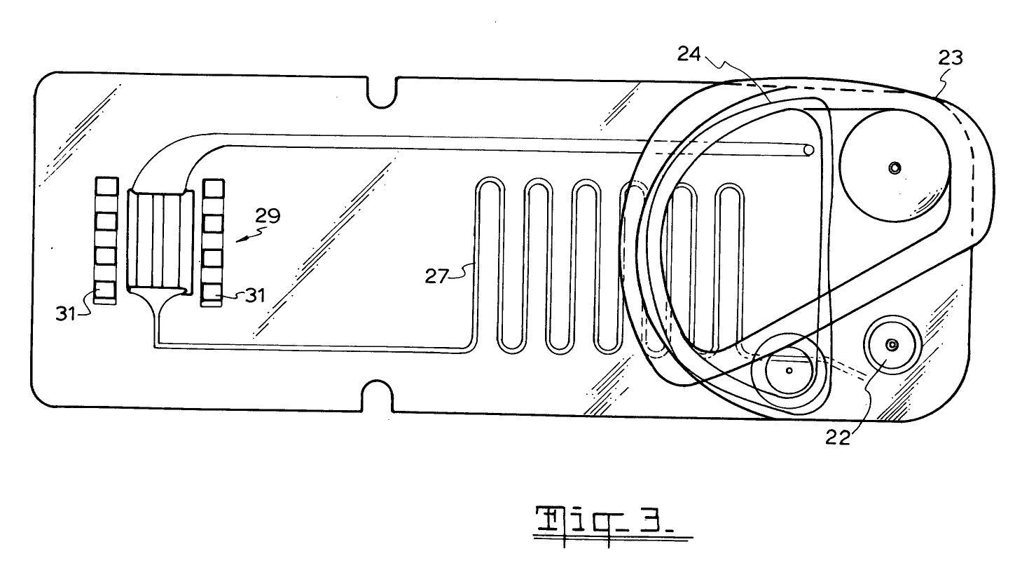

FIG. 3 illustrates a plan view of the fluid flow path of the detector unit.

FIG. 4 illustrates an exploded view of a second embodiment of this invention;

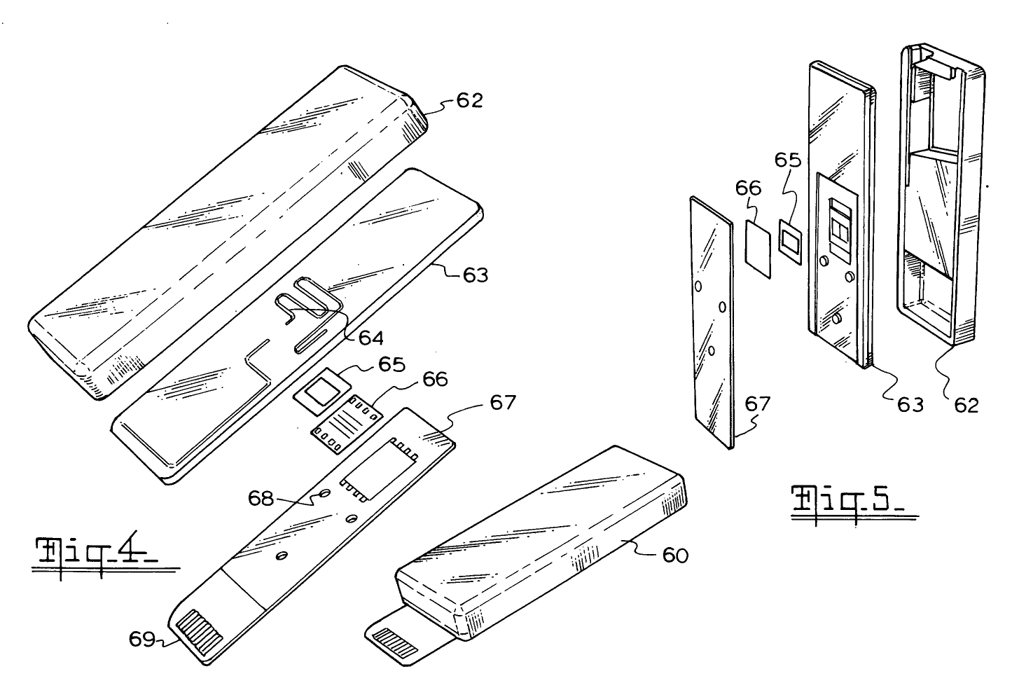

FIG. 5 is a reverse view of the embodiment of FIG. 4 shown in the operative orientation;

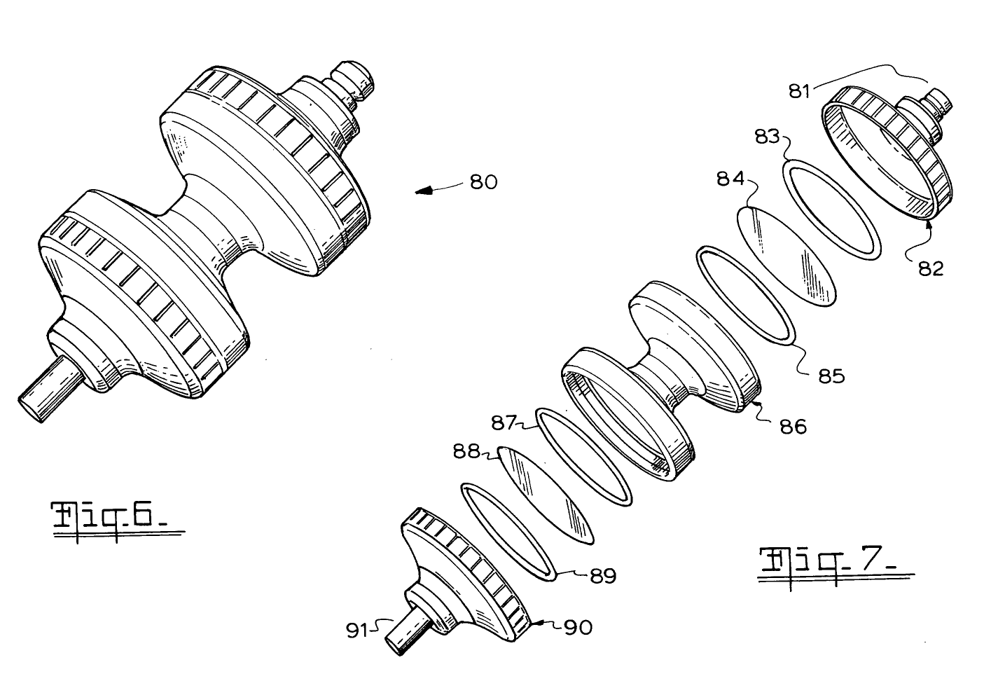

FIG. 6 illustrates the sample filtration unit of this invention;

FIG. 7 illustrates an exploded view of the sample filtration unit of FIG. 6.

The system consists of the detector unit 20 the reader unit 40 and a PDA 60.

The reader consists of a bottom unit 41 and a cover 42. the cover incorporates a docking bay 43 for the PDA 60 and also incorporates a slide button 44 for locking the detector unit 20 into electrical contact with the reader.

The docking recess 47 accommodates the detector unit 20 when it is slid into the recess. The spring hinged block 48 contains SAW printed circuit board (PCB) 49 for reading the SAW sensor on the detector unit. The SAW PCB contains spring pins for connection to the SAW sensor on the detection unit 20 as well as signal amplification circuitry and an interface to the main reader PCB 51. The reader PCB incorporates a battery such as a Lithium ion battery; a charging circuit; an interface to the SAW PCB 49; an interface to the PDA 60; and a USB interface for connection to an external PC if that is desired. The PDA 60 used in this embodiment is a Palm Tungsten T3.

The detector unit 20 includes a fluid reservoir 21 made from polymethylmethacrylate PMMA containing 3 separate reservoirs; namely a reservoir 22 for a blank solution of about 150 microlitres capacity, a sample reservoir 23 of about 3 ml capacity and a waste reservoir 24 of 2 ml which is located below the sample and blank reservoirs so that fluid flows under gravity from the blank and sample reservoirs through the microfluidic pathway to the waste reservoir.

The microfluidic layer 26 also fabricated from PMMA incorporates a serpentine microfluidic channel 27 on its bottom face and is designed to hold 45 seconds of blank solution flow.

The SAW device 29 incorporates electrical connections 31 to interface with the SAW PCB 49 and at least one saw device with a gold layer coated with a target specific agent. The saw device 29 is located within a recess of a frame layer 31 adhered by adhesive layer 25 to the microfluidic layer 26. The SAW device is preferably of the type disclosed in WO 02/95940 and Australian application 2004900942.

A base decal layer 33 is adhered to the cover layer to complete the detector unit. The detector unit 20 has the following functions.
Locates the SAW device in the reader
Allows for reliable electrical connection to the SAW PCB 49
Provides for a blank solution of distilled water to be collect the calibrating cells at point 28 and flow them over the SAW sensor 29
Provides for a sample solution from the sample reservoir to be passed over the SAW sensor 29
Provides for storage of the waste solutions

After the unit 20 has been inserted into the reader system 40, a clean buffered or blank solution is added to the ‘blank’ reservoir 22. This is fed by gravity and with the aid of capillary wicking flows through a long serpentine microchannel in layer 26 before passing over the SAW sensor 29 then to the ‘waste’ well 24 were the flow stops. The unit 20 is then left for between 1-24 hours to allow the SAW sensor 29 to soak. This step may not always be required. A controlled volume of sample is then added to the ‘sample’ reservoir 23. The sample flow is gravity fed into the same (blank filled) serpentine channel before flowing over the SAW sensor 29 and then to the waste reservoir 24. The amount of time the sample flows for is determined by the volume of the waste reservoir.

The purpose of the serpentine channel is to store blank solution. Once the sample is added, blank solution will flow over the SAW sensor 29 for a known period of time before the real sample comes into contact with it. This way, when data logging of the SAW response starts there will be up to 2 minutes preferably 45 seconds of blank or zero concentration flow before a frequency shift or response is detected. Once the frequency shift stops changing, the concentration of the target in the sample can be calculated by the software by subtracting the baseline zero level. This system does not require any external connections to provide pumping energy and results in a very reliable and repeatable flow regime. The flow rate diminishes over time, however this is very repeatable between uses.

In calibrating the sensor a known quantity of the non viable form of the target species may be placed in the microfluidic channel upstream of the SAW sensor. Distilled water from the blank solution is used to flush the non viable material and flow it across the sensor to obtain the first calibrating measurement. Only the quantity of the non viable material need be known not the volume of the distilled water.

The reservoirs are sized and positioned to maximise the available pressure head.

The serpentine channel is milled or moulded into a 2 mm thick piece of PMMA. The depth of the channel is 0.6 mm deep by 0.4 mm wide and about 500 mm long. This stores just under 1 minute of fluid flow. A three piece laminate is formed which holds the SAW sensor 29 in place and forms the flow channels. The flow cell containing the saw sensor is 5 mm wide and about 150 microns deep. Adhesive tape may be used to join and seal the layers together.

By packaging the SAW device in this way a lower cost, more fully integrated disposable solution is achieved.

In the embodiment illustrated in FIGS. 4 & 5 the detector unit 60 is designed to be inserted vertically in the reader unit. The vertical orientation ensures a faster flow rate and increases the head of the reservoir. The unit 60 consists of an injection moulded cover 62 incorporating the sample reservoir and waste reservoir and a fluidic channel section 63 incorporating the fluidic channels 64 arranged to flow sample over the SAW 66. The SAW device 66 is adhered to the microfluidic section 64 by the adhesive tape 65 and is wire bonded to the PCB carrier 67. The PCB carrier 67 incorporates assembly location holes 68 to correctly align the PCB circuit the SAW device 66 and the microfluidics 64. The PCB carrier 67 incorporates the contact pads 69 which connect to the memory stick reader in the reader unit. In most applications the sample needs to be filtered before its is passed through the microfluidic channels and over the SAW device. The filter removes the substances to be detected and allows the sample used in the detector to be a concentrated sample of the fluid being analysed. The filter unit 80 shown in FIGS. 6 and 7 consists of two filter units of a coarse and a fine pore size. The first filter units consists of a water inlet 81 having a screw threaded housing 82 that connects to one end of the dual screw threaded central housing 86. Between the units 82 and 86 is positioned a 1-3 micron filter sealed on each side by rubber O rings 83 and 85. The second filter is a 2 to O.45 micron filter held between O rings seals 87 and 89. The fluid to be tested is passed through the filter unit utilising the pressure of the fluid itself which is usually under a pressure head for example in an airconditioning cooling tower or under pumping pressure as in a processing fluid. The pressure ensures that sufficient volume of fluid passes through the filter to enable a sufficient amount of the targeted biological species to be collected. The us of mains pressure enables a litre of fluid to be filtered in less than a minute compared to over an hour if gravity alone is relied upon. The filters are rinsed with blank solution to provide a concentrated sample for the detector unit.

Those skilled in the art will realise that other embodiments are possible without departing from the core teachings of this invention.

1. A sensor system for analyzing fluid samples for the presence of a target biological or chemical species comprising a
a) a detector unit consisting of a body incorporating a surface acoustic wave (SAW) sensor for said target species, disposed in a microfluidic channel and a storage reservoir for processed fluid
b) a reader unit adapted to receive the detector unit said reader unit including means to establish electrical contact with said surface acoustic wave sensor and a processor to analyse the sensor signals to determine if the target species is in said sample.

2. A sensor as claimed in claim 1 in which the surface acoustic wave sensor is incorporated in the reader unit and the detector unit incorporates a biologically sensitive surface that seats on the surface acoustic wave sensor when the detector unit is docked in the reader unit.

3. A sensor as claimed in claim 1 in which the detector unit includes a storage reservoir for the sample to be tested arranged so that it is located above the storage reservoir for the treated sample so that flow through the microfluidic channel occurs under the influence of gravity.

4. A sensor system as claimed in claim 1 in which the surface acoustic wave sensor incorporates
a) a first layered SAW device consisting of a piezoelectric crystal with interdigital electrodes on its surface, and second piezoelectric layer over said interdigital electrodes
b) a second layered SAW device consisting of a piezoelectric crystal with interdigital electrodes on its surface, a second piezoelectric layer over said interdigital electrodes and a biologically sensitive surface on said second piezoelectric layer
wherein both SAW devices are fabricated on the same substrate.

5. A sensor system as claimed in claim 3 in which the biologically sensitive surface contains a reagent that binds to the target species.

6. A sensor system as claimed in claim 4 in which the target species is Salmonella, E Coli or legionella.

7. A detector unit for use in the sensor system defined in claim 1 consisting of a body having a sample reservoir and a microfluidic channel leading to a storage reservoir for processed fluid said body incorporating a surface acoustic wave (SAW) sensor for said target species disposed in said microfluidic channel the arrangement being that the sample fluid flows under gravity from the sample reservoir to the waste reservoir.

8. A detector unit as claimed in claim 7 in which the SAW sensor is a layered SAW device consisting of a piezoelectric layer said interdigital electrodes and a biologically sensitive surface on said second piezoelectric layer.

9. A method of calibrating a biological sensor of the type in which a sample fluid containing a target species is brought into contact with a receptor surface containing a reagent that binds to the target species the calibration technique including the steps of contacting the receptor surface with first liquid having a known quantity of a form of the target species to provide a first measurement and then contacting the receptor with the sample solution to be measured and taking a second measurement and using the first measurement to calibrate the sensor quantitatively to determine the quantity of the target in the sample solution from the second measurement.

10. A method as claimed in claim 9 in which the sensor consists of a biological sensitive surface over a surface acoustic wave sensor and the flow of calibrating solution over the sensor surface produces a first frequency change and the sample solution produces a second frequency change.

11. A sample collection device for use in preparing a sample for the detector unit defined in claim 1 which includes
a) a first housing having a fluid inlet and a filter having a pore size to retain target biological species said housing having an outlet
b) a second housing connected to the outlet of said first housing having a filter having a pore size to retain said target biological species said second hosing having an outlet to a waste receptacle.
